(19) United States
(12) Patent Application Publication
(10) Pub. No.: US 2026/0199696 A1
(43) Pub. Date: Jul. 16, 2026

(54) DUAL-CONFIGURED POWER FOR AN AUTOMATED EXTERNAL DEFIBRILLATOR

(71) Applicant: Toyota Motor Engineering & Manufacturing North America, Inc., Plano, TX (US)

(72) Inventor: Ercan Mehmet Dede, Ann Arbor, MI (US)

(73) Assignee: Toyota Motor Engineering & Manufacturing North America, Inc., Plano, TX (US)

(21) Appl. No.: 19/016,071

(22) Filed: Jan. 10, 2025

(51) Int. Cl. A61N 1/39; A61N 1/36

(52) U.S. Cl. CPC A61N 1/3981; A61N 1/3603; A61N 1/3904; A61N 1/3912

(57) ABSTRACT

Systems and methods described herein relate to determining if a low-voltage power component lacks power to generate an electric shock signal and generating the electric shock signal via a high-voltage power component if the low-voltage power component lacks power to generate the electric shock signal. For example, if a rechargeable battery fails within an automatic external defibrillator, an electric shock signal may be generated from a high-voltage power supply such as a high-voltage electric vehicle battery.

### TECHNICAL FIELD

The subject matter described herein relates, in general, to strategies for managing automated external defibrillators in a vehicular environment.

### BACKGROUND

An automated electronic defibrillator (AED) is an electronic device that is used to restore normal heart rhythm to a patient that is experiencing a cardiac event. During a cardiac event, conductive pads placed on a patient allow an AED to detect and analyze the patient's heart rhythm. If the AED detects an abnormal rhythm, such as ventricular fibrillation or tachycardia, the AED may then deliver a high voltage shock, typically 200 to 1000 volts, through the electrodes of the pads to the heart. This electric shock may halt the arrhythmia, after which the heart may resume a normal rhythm.

### SUMMARY

In one embodiment, a vehicle management system is disclosed. The vehicle management system includes one or more processors and a memory communicably coupled to the one or more processors. The memory stores a command module including instructions that when executed by the one or more processors cause the one or more processors to determine if a low-voltage power component lacks power to generate an electric shock signal and generate the electric shock signal via a high-voltage power component if the low-voltage power component lacks power to generate the electric shock signal.

In one embodiment, a non-transitory computer-readable medium including instructions that when executed by one or more processors cause the one or more processors to perform one or more functions is disclosed. The instructions include instructions to determine if a low-voltage power component lacks power to generate an electric shock signal and generate the electric shock signal via a high-voltage power component if the low-voltage power component lacks power to generate the electric shock signal.

In one embodiment, a method is disclosed. In one embodiment, the method includes determining if a low-voltage power component lacks power to generate an electric shock signal and generating the electric shock signal via a high-voltage power component if the low-voltage power component lacks power to generate the electric shock signal.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

FIG. 1 illustrates one embodiment of a vehicle within which systems and methods disclosed herein may be implemented.

FIG. 2 illustrates one embodiment of an AED management system that is associated with managing an AED in a vehicular environment.

FIG. 3 illustrates one example of an AED system that may operate within vehicle 100 as part of an AED management system.

FIG. 4 illustrates one example of a typical low-voltage shock signal generator.

FIG. 5 illustrates one example of a method for managing an AED in a vehicular environment.

### DETAILED DESCRIPTION

Systems, methods, and other embodiments associated with AED management are described herein. AEDs typically utilize a low-voltage circuit to generate an electric shock signal. However, such a low-voltage circuit may lack power to generate such an electric shock signal (e.g., due to an expired or damaged rechargeable battery, a defective battery charger, a defective AED charging circuit, etc.).

In situations where the low-voltage battery system cannot provide a high-voltage shock signal, the AED management system described herein may provide the ability to generate a high-voltage shock signal from other power sources, such as the high-voltage power available from an electric or hybrid vehicle battery.

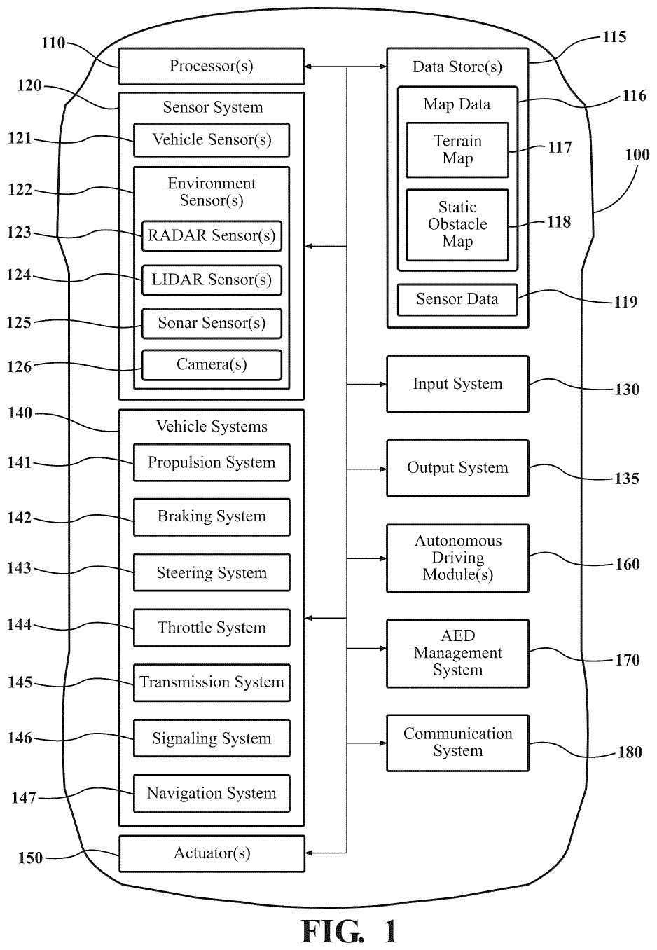

Referring to FIG. 1, an example of a vehicle 100 is illustrated. As used herein, a “vehicle” is any form of motorized transport. In one or more implementations, vehicle 100 is an automobile. While arrangements will be described herein with respect to automobiles, it will be understood that embodiments are not limited to automobiles. In some implementations, vehicle 100 may be any robotic device or form of motorized transport that, for example, includes sensors to perceive aspects of the surrounding environment, and thus benefits from the functionality discussed herein associated with diagnostic charging strategies. As a further note, this disclosure generally discusses vehicle 100 as traveling on a roadway with surrounding vehicles, which are intended to be construed in a similar manner as vehicle 100 itself. That is, the surrounding vehicles may include any vehicle that may be encountered on a roadway by vehicle 100.

Vehicle 100 also includes various elements. It will be understood that in various embodiments it may not be necessary for vehicle 100 to have all of the elements shown in FIG. 1. Vehicle 100 may have any combination of the various elements shown in FIG. 1. Further, vehicle 100 may have additional elements to those shown in FIG. 1. In some arrangements, vehicle 100 may be implemented without one or more of the elements shown in FIG. 1. While the various elements are shown as being located within vehicle 100 in FIG. 1, it will be understood that one or more of these elements may be located external to vehicle 100. Further, the elements shown may be physically separated by large distances. For example, as discussed, one or more components of the disclosed system may be implemented within a vehicle while further components of the system are implemented within a cloud-computing environment or other system that is remote from vehicle 100.

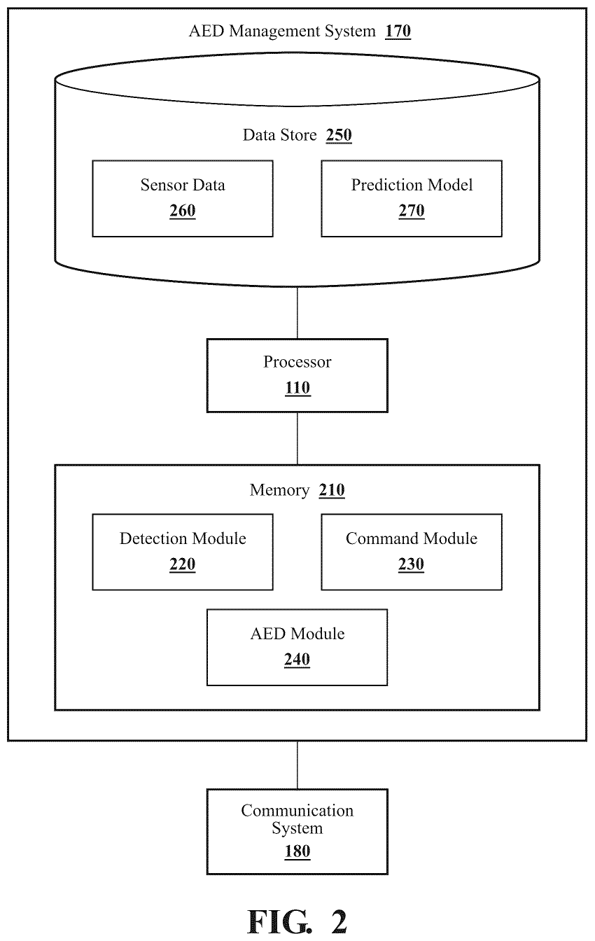

Some of the possible elements of vehicle 100 are shown in FIG. 1 and will be described along with subsequent figures. However, a description of many of the elements in FIG. 1 will be provided after the discussion of FIGS. 2-5 for purposes of brevity of this description. Additionally, it will be appreciated that for simplicity and clarity of illustration, where appropriate, reference numerals have been repeated among the different figures to indicate corresponding or analogous elements. In addition, the discussion outlines numerous specific details to provide a thorough understanding of the embodiments described herein. Those of skill in the art, however, will understand that the embodiments described herein may be practiced using various combinations of these elements. In either case, vehicle 100 includes an AED management system 170 that is implemented to perform methods and other functions as disclosed herein. As will be discussed in greater detail subsequently, AED management system 170, in various embodiments, is implemented partially within vehicle 100 and as a cloud-based service. For example, in one approach, functionality associated with at least one module of AED management system 170 is implemented within vehicle 100 while further functionality is implemented within a cloud-based computing system.

With reference to FIG. 2, one embodiment of AED management system 170 of FIG. 1 is further illustrated. AED management system 170 is shown as including processors 110 from vehicle 100 of FIG. 1. Accordingly, processors 110 may be a part of AED management system 170, AED management system 170 may include a separate processor from processors 110 of vehicle 100, or AED management system 170 may access processors 110 through a data bus or another communication path. In one embodiment, AED management system 170 includes memory 210, which stores detection module 220 and command module 230. Memory 210 is a random-access memory (RAM), read-only memory (ROM), a hard-disk drive, a flash memory, or other suitable memory for storing detection module 220 and command module 230. Detection module 220 and command module 230 are, for example, computer-readable instructions that when executed by processors 110 cause processors 110 to perform the various functions disclosed herein.

AED management system 170 as illustrated in FIG. 2 is generally an abstracted form of AED management system 170 as may be implemented between vehicle 100 and a cloud-computing environment. Accordingly, AED management system 170 may be embodied at least in part within a cloud-computing environment to perform the methods described herein.

With reference to FIG. 2, detection module 220 generally includes instructions that function to control processors 110 to receive data inputs from one or more sensors of vehicle 100. The inputs are, in one embodiment, observations of one or more objects in an environment proximate to vehicle 100, other aspects about the surroundings, or both. As provided for herein, detection module 220, in one embodiment, acquires sensor data 260 that includes at least camera images. In further arrangements, detection module 220 acquires sensor data 260 from further sensors such as radar 123, LiDAR 124, and other sensors as may be suitable for identifying vehicles, locations of the vehicles, lane markers, crosswalks, traffic signs, vehicle parking areas, road surface types, curbs, vehicle barriers, and so on.

In one embodiment, detection module 220 may also acquire sensor data 260 from one or more sensors that allows for the detection of load characteristics for a load that will be transported by a vehicle or trailer. For example, load data may be comprised of any sensor data 260 that may be relevant to the determination of the size (e.g., height, width, length), weight, density, or any other static or dynamic property of a load that may affect vehicle operation before, during, or after transport. A load may be any form of cargo or freight that is transported on a trailer, on a vehicle (e.g., in a pickup truck bed), or as a detachable part of a trailer or vehicle.

Accordingly, detection module 220, in one embodiment, controls the respective sensors to provide sensor data 260. Additionally, while detection module 220 is discussed as controlling the various sensors to provide sensor data 260, in one or more embodiments, detection module 220 may employ other techniques to acquire sensor data 260 that are either active or passive. For example, detection module 220 may passively sniff sensor data 260 from a stream of electronic information provided by the various sensors to further components within vehicle 100. Moreover, detection module 220 may undertake various approaches to fuse data from multiple sensors when providing sensor data 260, from sensor data acquired over a wireless communication link from one or more of the surrounding vehicles or other sources (e.g., via V2V, V2I, V2X), or from a combination thereof. Thus, sensor data 260, in one embodiment, represents a combination of perceptions acquired from multiple sensors.

In addition to locations of surrounding vehicles, sensor data 260 may also include, for example, odometry information, GPS data, or other location data. Moreover, detection module 220, in one embodiment, controls the sensors to acquire sensor data about an area that encompasses 360 degrees about vehicle 100, which may then be stored in sensor data 260. In some embodiments, such area sensor data may be used to provide a comprehensive assessment of the surrounding environment around vehicle 100. Of course, in alternative embodiments, detection module 220 may acquire the sensor data about a forward direction alone when, for example, vehicle 100 is not equipped with further sensors to include additional regions about the vehicle or the additional regions are not scanned due to other reasons (e.g., unnecessary due to known current conditions).

Moreover, in one embodiment, AED management system 170 includes a database 250. Database 250 is, in one embodiment, an electronic data structure stored in memory 210 or another data store and that is configured with routines that may be executed by processors 110 for analyzing stored data, providing stored data, organizing stored data, and so on. Thus, in one embodiment, database 250 stores data used by the detection module 220 and command module 230 in executing various functions. In one embodiment, database 250 includes sensor data 260 along with, for example, metadata that characterize various aspects of sensor data 260. For example, the metadata may include location coordinates (e.g., longitude and latitude), relative map coordinates or tile identifiers, time/date stamps from when separate sensor data 260 was generated, and so on.

Detection module 220, in one embodiment, is further configured to perform additional tasks beyond controlling the respective sensors to acquire and provide sensor data 260. For example, detection module 220 includes instructions that may cause processors 110 to obtain load characteristics as described herein. In some embodiments, detection module 220 may receive and store load characteristics.

In some embodiments, command module 230 generally includes instructions that function to control the processors 110 or collection of processors in a cloud-computing environment.

In some embodiments, command module 230 may provide any vehicle functionality needed to support AED module 240. For example, command module 230 may cause vehicle 100 to provide power functions enabling the functionality provided by AED module 240. As another example, command module 230 may provide a communication services via vehicle 100 to facilitate communication by AED module 240 as described herein.

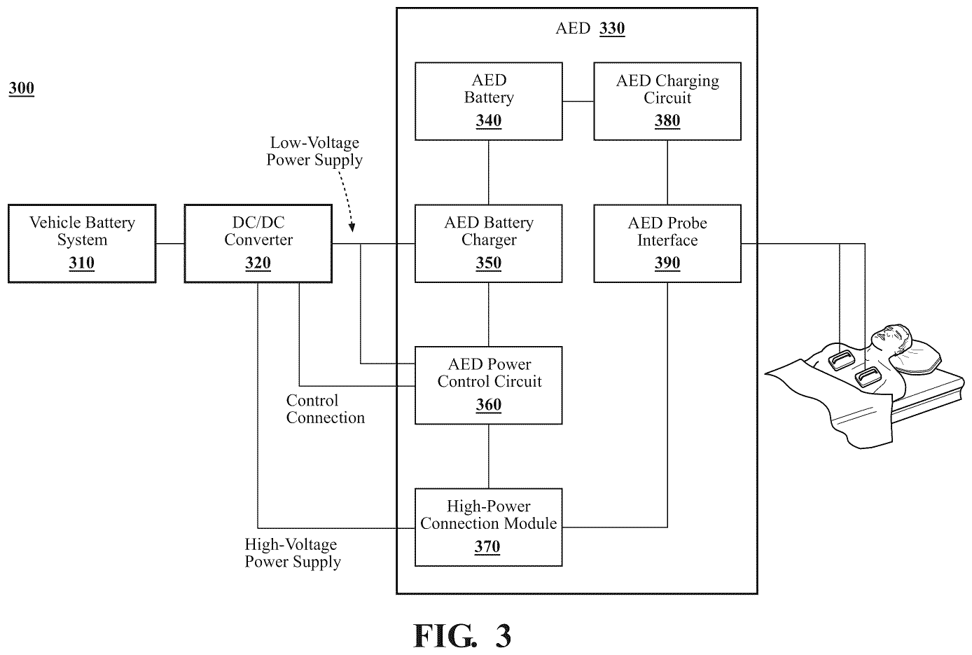

In some embodiments, and with reference to FIG. 2 and FIG. 3, AED module 240 may provide full or partial functionality as described with respect to AED management system 300. While the example that follows is described from the perspective that: (a) command module 230 may control vehicle battery system 310 and DC/DC converter 320 to support AED module 240; and (b) AED module 240 may control AED 330, AED battery 340, AED battery charger 350, AED power control circuit 360, high-power connection module 370, AED charging circuit 380, and AED probe interface 390, it should be understood that in some embodiments command module 230 or AED module 240 may individually or collectively control various aspects of AED management system 300. With respect to FIG. 3, an example of an AED management system 300 is shown that may be part of vehicle 100 within AED management system 170. Vehicle battery system 310 may be a high-voltage battery pack of an electrical vehicle, hybrid vehicle, or fuel-cell vehicle (e.g., as part of vehicle propulsion system 141). Due to their high-voltage operation, typically around 400 or 800 volts, high-voltage battery packs are often encased in an aluminum or high-strength composite within the internal structure of the vehicle. Furthermore, electrical protection for the high-voltage battery may be provided by isolating the vehicle from the chassis, using fuses or circuit breakers capable of cutting off power in case of a fault, and a battery management system that monitors the state of the battery pack and prevents dangerous charging/discharging or other undesired activities. High-voltage battery packs also often encompass additional safety features such as integrated cooling/heating, environmental protection (e.g., waterproofing), and so on.

Given that access to the battery power of the high-voltage battery pack is considerably more restricted than battery power from traditional lead-acid battery systems, electrical vehicles or hybrid vehicles often utilize a DC/DC converter 320 to generate a lower 12 volt or 48 volt power supply for vehicle systems or auxiliary devices connected to the vehicle. As shown in FIG. 3, DC/DC converter 320 may provide a low-voltage power supply (e.g., via buck converter functionality) for vehicle systems or auxiliary devices connected to the vehicle. However, unlike traditional DC/DC converters, DC/DC converter 320 may also provide a high-voltage output higher than the output voltage of Vehicle Battery System 310 (e.g., via boost converter functionality, or both) that may be connected to AED 330.

AED 330 is one or more AEDs that may be connected to or incorporated within vehicle 100. For example, AED 330 may be concealed within a dashboard until access is requested, after which it may be revealed for use. In some embodiments, access may be requested by AED 330 if a potential cardiac event is present. For example, AED 330 may monitor sensor data 260 for information indicating a potential cardiac event, such as electrocardiogram data (e.g., ECG or EKG taken by an electrical heart sensor), images or videos of the driver, control inputs (e.g., sudden erratic behavior), electroencephalogram(s) (EEGs), audio recordings (e.g., voice patterns, breathing patterns), eye movements (e.g., uncontrolled eye movements, eye rollback), and so on. In some embodiments, AED 330 may also receive information indicating a potential cardiac event from devices connected to vehicle 100, such as wearables (e.g., smart watches), pacemakers, etc. In some embodiments, AED 330 may utilize neural networks or other deep learning techniques to analyze sensor data 260 (which may also include data from other sources such as smart watches and pacemakers) and determine if a potential cardiac event is present.

In some embodiments, AED 330 may receive an instruction that a potential cardiac event is present, such as by action of a vehicle operator or passenger instructing vehicle 100 to provide access to AED 330 (e.g., via input system 130). As another example, a remote operator (e.g., 911 dispatch operator) may send an instruction that a potential cardiac event is present to AED 330, such as by a wireless communication to the vehicle (e.g., via vehicle-to-vehicle, vehicle-to-infrastructure, vehicle-to-everything, a cloud computing environment).

In some embodiments, upon receiving a request to access AED 330, AED 330 may generate an authentication request (e.g., multifactor authentication) requiring at least one validation action that validates the access request. For example, an authentication request may instruct a person to present a passcode (e.g., a number known to the vehicle operator/occupant or transferred to a device associated with the vehicle operator/occupant) to vehicle 100 in order to validate an access request. As another example, an authentication request may instruct a person to present themselves in a manner allowing for biometric identification (e.g., facial identification, fingerprint identification) by vehicle 100, which may then be checked to ensure the person is authorized to request access. As yet another example, an authentication request may provide notification (e.g., via vehicle 100) that an access request to AED 330 has been presented and allow for a user to reject the validity of such a request.

In some embodiments, AED 330 may contain different authentication requests for different circumstances. For example, AED 330 may utilize passcode verification only when a cardiac event is determined not to be present, such as to avoid undesirable operation of AED 330 by children or theft of AED 330. As another example, when a cardiac event is determined to be present, AED 330 may provide an authentication request providing notification that operation of the AED system is being recorded and will be sent to emergency responders unless a user cancels the access request, which if such cancellation is not performed results in the access request being validated.

In some embodiments, AED 330 may provide communication of all information pertaining to the detection of a potential cardiac event, the operation of the AED 330 to analyze a patient or administer treatment, and so on to a third party, such as an emergency medical service. Such communication may occur for example by wireless communication between vehicle 100 to a third-party server.

In some embodiments, AED 330 may receive or generate an instruction to suspend one or more operations of AED 330. For example, if AED 330 is accessed or removed in an unauthorized manner, AED 330 may be instructed to deny access to one or more capabilities until as such time as a proper authorization is received by AED 330 to restore such capabilities. As another example, AED 330 may receive an instruction to cease providing a capability because it is no longer necessary (e.g., patient death) or misuse (e.g., criminal abuse), such as through instructions sent by emergency responders remotely observing activities within vehicle 100.

In some embodiments, AED 330 may provide medical privacy protection as required by law (e.g., HIPAA). Accordingly, a user may be required to authorize AED 330 to be able to provide medical information to a third party prior to AED 330 performing such an action.

In some embodiments, AED battery 340 of AED 330 may be maintained by AED battery charger 350 coupled to DC/DC converter 320. For example, AED battery charger 350 may be coupled to a standard 12 volt or 48 volt power supply provided by DC/DC converter 320. AED battery charger 350 may maintain AED battery 340 and also perform diagnostic tests ensuring that AED battery 340 is not defective or otherwise impaired (e.g., depleted from prior use of AED 330). If AED battery charger 350 determines that AED battery 340 is not in a condition to allow AED system to operate properly, AED battery charger 350 may indicate (e.g., via a control signal) to AED power control circuit 360 that AED battery 340 is inoperative. In some embodiments, AED battery charger 350 may also provide information to AED power control circuit 360 indicating whether AED battery charger 350 is operating correctly.

In some embodiments, AED power control circuit 360 may allow AED 330 to access high-voltage power via DC/DC converter 320, such as upon receiving an indication that AED battery 340 or AED battery charger 350 is inoperative. For example, AED power control circuit 360 may cause DC/DC converter 320 to provide a temporary high-power connection to high-power connection module 370, thus allowing AED 330 to obtain high-voltage power (e.g., 200 volts, 400 volts, 800 volts) directly from an output of DC/DC converter 320 that is above a standard voltage output provided by DC/DC converter 320 (e.g., 12 volts, 48 volts). As another example, if DC/DC converter 320 regularly provides a high-power connection to high-power connection module 370, AED power control circuit 360 may switch on or off high-power connection module 370 (e.g., via a control connection between AED power control circuit 360 and high-power connection module 370) to obtain power for AED 330 as needed.

In some embodiments, high-power connection module 370 of AED 330 may operate to condition high-voltage power for use by AED 330. For example, if AED 330 cannot receive normal operating power (e.g., because of a failure arising in from AED battery 340 or AED battery charger 350; a lack of low-voltage power supply from DC/DC Converter 320), high-power connection module 370 may provide backup operating power to AED 330 until such time as normal operating power is restored. For example, AED power control circuit 360 may instruct high-power connection module 370 to provide the desired backup operating power for AED 330 from a high-voltage connection (e.g., connection of AED 330 to a high-voltage output of DC/DC converter 320), such as via buck converter functionality within high-power connection module 370. In some embodiments, the desired backup operating power may be same voltage as the low-voltage power supply (e.g., when supplied to AED battery charger 350 from high-power connection module 370). In some embodiments, the desired backup operating power may be the same voltage the output of AED battery charger 350 (e.g., when supplied to AED battery 340 from high-power connection module 370). In some embodiments, the desired backup operating power may be the same voltage as the voltage output of the AED battery 340.

In some embodiments, if AED 330 has insufficient power to provide high voltage shocks (e.g., because the battery is too depleted, failure in AED charging circuit 380), high-power connection module 370 may condition the high-voltage power that it receives to form a high-voltage electric shock signal. In some embodiments, providing a high-voltage electric shock signal from a high-voltage DC input may be performed by DC/DC converter 320 (e.g., where high-power connection module 370 acts as a pass-through), by high-power connection module 370, or a combination of both (e.g., DC/DC converter supplies a high voltage supply as specified by AED 330, such as via AED power control circuit 360, to high-power connection module 370, after which high-power connection module 370 modulates the high-voltage shock signal). In some embodiments, the instruction to provide the high-voltage electric shock signal from a high-voltage DC input may be provided by AED 330 (e.g., via AED power control circuit 360 if it detects insufficient low-voltage power, such as due to a battery failure), DC/DC converter 320 (e.g., where it receives information about a failure in AED 330, such as an inability to charge or a fault signal), or other modules within vehicle 100. For example, AED 330 may be configured such that DC/DC converter 320 can pass a high-voltage shock signal to AED probe interface 390 through high-power connection module 370 if AED 300 enters a failed state.

AED probe interface 390 may provide an interface for any cables that may be attached to AED 330, such as electrodes for analyzing a heart's electrical activity, delivering a high-voltage electric shock signal, etc. For example, AED probe interface 390 may provide an interface for therapy cables that connect AED 330 to electrode pads that are placed on a patient's chests; ECG cables that connect to ECG electrodes; training cables for practice sessions; USB or other well-known connectors; and so on.

In some embodiments, AED 330 may adjust the generation of a high-voltage electric signal based on patient information. For example, DC/DC converter 320, high-power connection module 370, or AED charging circuit may reduce the intensity of a high-voltage electric signal for sensitive patients (e.g., children).

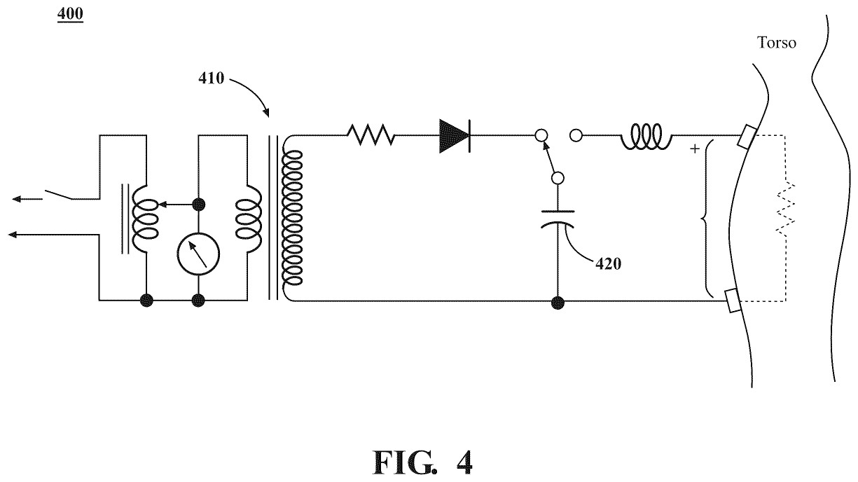

As shown in FIG. 4, an example of AED charging circuit 380 for providing high-voltage shocks is shown. AED charging circuit 380 may utilize step-up transformer 410 to boost a low-voltage power supply (e.g., 9 to 15 volts from a rechargeable battery) to a much higher voltage (e.g., hundreds of volts), which is then used to store energy in capacitor 420. In some embodiments, when AED 330 determines that a shock is needed (e.g., via AED power control circuit), the discharge of capacitor 420 after having been charged by step-up transformer 410 supplies the high-voltage electric shock signal from AED charging circuit 380 to AED probe interface 390.

With respect to AED system and methods described herein, the ability to access a high-voltage power supply (e.g., 400 volts, 800s volts) allows for new approaches that may be utilized to condition a high-voltage power supply to provide electric shock signals (e.g., via DC/DC converter 320 or high-power connection module 370). For example, metal-oxide field-effect transistors (MOSFETs), insulated-gate bipolar transistors (IGBTs), or both may be used to former inverter circuits that condition a high-voltage power supply to provide electric shock signals with appropriate monophasic or biphasic waveform. Such conditioning circuits may also utilize wideband gap materials better suited for high-power, high frequency applications, such as gallium nitride (GaN) and silicon carbide (SiC). Accordingly, DC/DC converter 320 or high-power connection module 370 may utilize MOSFETs, IGBTs, or a combination thereof to generate high-voltage pulse width modulated signals of an appropriate waveform that may be used to provide a high-voltage electric shock signal (e.g., via AED probe interface 390). In some embodiments, DC/DC converter 320 or high-power connection module 370 may utilize an energy storage capacitor similar to the capacitor 420 shown in FIG. 4 or other suitable capacitors in the electric, hybrid, or fuel cell vehicle, such as capacitors found in a vehicle power control unit (not shown).

In some embodiments, AED management system 170 may be implemented as described herein in a non-vehicular environment. For example, as opposed to a high-voltage vehicle battery, a high-voltage power source may be obtained from a generator or home battery system. In some embodiments, multiple instances of AED 330 may be implemented as described herein with respect to a high-voltage power source (e.g., an ambulance, a temporary hospital). In some embodiments, AED management system 170 may utilize a portable AED device as AED 330 when the portable AED device is coupled to vehicle 100.

In some embodiments, AED module 240 may instruct a person in how to apply conductive pads to a patient. In some embodiments, AED module 240 may analyze images of a patient to determine one or more locations to apply conductive pads, display where such locations are to a user (e.g., on multimedia devices in the vehicle, through diagrams transmitted to mobile devices), and correct a user if one or conductive pads are observed to be applied incorrectly. In some embodiments, AED module 240 may instruct an apparatus coupled to vehicle 100 (e.g., via command module 230) to remove clothing and apply one or more conductive pads. For example, based on having determined one or more locations to apply conductive pads, AED module 240 may instruct a robotic arm to grip and pull clothing at such a location, rotate a cutting element in a circular motion around the pulled clothing, and then apply a conductive pad to the skin of a patient. In some embodiments, AED module 240 may instruct vehicle 100 to apply restraints to a vehicle occupant, such as tightening a seatbelt to restrain the movements of the vehicle occupant.

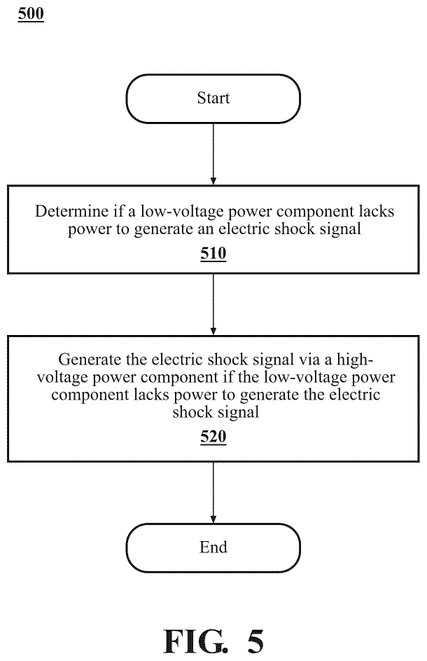

FIG. 5 illustrates a flowchart of a method 500 that is associated with using load management strategies. Method 500 will be discussed from the perspective of the AED management system 170 of FIGS. 1 and 2. While method 500 is discussed in combination with the AED management system 170, it should be appreciated that the method 500 is not limited to being implemented within AED management system 170 but is instead one example of a system that may implement method 500.

At step 510, AED module 240 may determine if a low-voltage power component lacks power to generate an electric shock signal. For example, AED module 240 may detect a battery failure, charger failure, circuit failure, or other condition that prevents use of a low-voltage power component to generate an electric shock signal.

At step 520, AED module 240 may generate the electric shock signal via a high-voltage power component if the low-voltage power component lacks power to generate the electric shock signal. For example, if AED module 240 detects that low-voltage power component lacks power to generate the electric shock signal, it may instruct DC/DC converter 320 to provide high-voltage power to high-power connection module 370. High-power connection module 370 may then generate the electric shock signal using the high-voltage power, such as through an inverter circuit using wideband gap materials.

FIG. 1 will now be discussed in full detail as an example environment within which the system and methods disclosed herein may operate. In some instances, vehicle 100 is configured to switch selectively between various modes, such as an autonomous mode, one or more semi-autonomous operational modes, a manual mode, etc. Such switching may be implemented in a suitable manner, now known, or later developed. “Manual mode” means that all of or a majority of the navigation/maneuvering of the vehicle is performed according to inputs received from a user (e.g., human driver). In one or more arrangements, vehicle 100 may be a conventional vehicle that is configured to operate in only a manual mode.

In one or more embodiments, vehicle 100 is an autonomous vehicle. As used herein, “autonomous vehicle” refers to a vehicle that operates in an autonomous mode. “Autonomous mode” refers to using one or more computing systems to control vehicle 100, such as providing navigation/maneuvering of vehicle 100 along a travel route, with minimal or no input from a human driver. In one or more embodiments, vehicle 100 is either highly automated or completely automated. In one embodiment, vehicle 100 is configured with one or more semi-autonomous operational modes in which one or more computing systems perform a portion of the navigation/maneuvering of the vehicle along a travel route, and a vehicle operator (i.e., driver) provides inputs to the vehicle to perform a portion of the navigation/maneuvering of vehicle 100 along a travel route.

Vehicle 100 may include one or more processors 110. In one or more arrangements, processor(s) 110 may be a main processor of vehicle 100. For instance, processor(s) 110 may be an electronic control unit (ECU). Vehicle 100 may include one or more data stores 115 for storing one or more types of data. Data store(s) 115 may include volatile memory, non-volatile memory, or both. Examples of suitable data store(s) 115 include RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. Data store(s) 115 may be a component of processor(s) 110, or data store 115 may be operatively connected to processor(s) 110 for use thereby. The term “operatively connected,” as used throughout this description, may include direct or indirect connections, including connections without direct physical contact.

In one or more arrangements, data store(s) 115 may include map data 116. Map data 116 may include maps of one or more geographic areas. In some instances, map data 116 may include information or data on roads, traffic control devices, road markings, structures, features, landmarks, or any combination thereof in the one or more geographic areas. Map data 116 may be in any suitable form. In some instances, map data 116 may include aerial views of an area. In some instances, map data 116 may include ground views of an area, including 360-degree ground views. Map data 116 may include measurements, dimensions, distances, information, or any combination thereof for one or more items included in map data 116. Map data 116 may also include measurements, dimensions, distances, information, or any combination thereof relative to other items included in map data 116. Map data 116 may include a digital map with information about road geometry. Map data 116 may be high quality, highly detailed, or both.

In one or more arrangements, map data 116 may include one or more terrain maps 117. Terrain map(s) 117 may include information about the ground, terrain, roads, surfaces, other features, or any combination thereof of one or more geographic areas. Terrain map(s) 117 may include elevation data in the one or more geographic areas. Terrain map(s) 117 may be high quality, highly detailed, or both. Terrain map(s) 117 may define one or more ground surfaces, which may include paved roads, unpaved roads, land, and other things that define a ground surface.

In one or more arrangements, map data 116 may include one or more static obstacle maps 118. Static obstacle map(s) 118 may include information about one or more static obstacles located within one or more geographic areas. A “static obstacle” is a physical object whose position does not change or substantially change over a period of time and whose size does not change or substantially change over a period of time. Examples of static obstacles include trees, buildings, curbs, fences, railings, medians, utility poles, statues, monuments, signs, benches, furniture, mailboxes, large rocks, hills. The static obstacles may be objects that extend above ground level. The one or more static obstacles included in static obstacle map(s) 118 may have location data, size data, dimension data, material data, other data, or any combination thereof, associated with it. Static obstacle map(s) 118 may include measurements, dimensions, distances, information, or any combination thereof for one or more static obstacles. Static obstacle map(s) 118 may be high quality, highly detailed, or both. Static obstacle map(s) 118 may be updated to reflect changes within a mapped area.

Data store(s) 115 may include sensor data 119. In this context, “sensor data” means any information about the sensors that vehicle 100 is equipped with, including the capabilities and other information about such sensors. As will be explained below, vehicle 100 may include sensor system 120. Sensor data 119 may relate to one or more sensors of sensor system 120. As an example, in one or more arrangements, sensor data 119 may include information on one or more LIDAR sensors 124 of sensor system 120.

In some instances, at least a portion of map data 116 or sensor data 119 may be located in data stores(s) 115 located onboard vehicle 100. Alternatively, or in addition, at least a portion of map data 116 or sensor data 119 may be located in data stores(s) 115 that are located remotely from vehicle 100.

As noted above, vehicle 100 may include sensor system 120. Sensor system 120 may include one or more sensors. “Sensor” means any device, component, or system that may detect or sense something. The one or more sensors may be configured to sense, detect, or perform both in real-time. As used herein, the term “real-time” means a level of processing responsiveness that a user or system senses as sufficiently immediate for a particular process or determination to be made, or that enables the processor to keep up with some external process.

In arrangements in which sensor system 120 includes a plurality of sensors, the sensors may work independently from each other. Alternatively, two or more of the sensors may work in combination with each other. In such an embodiment, the two or more sensors may form a sensor network. Sensor system 120, the one or more sensors, or both may be operatively connected to processor(s) 110, data store(s) 115, another element of vehicle 100 (including any of the elements shown in FIG. 1), or any combination thereof. Sensor system 120 may acquire data of at least a portion of the external environment of vehicle 100 (e.g., nearby vehicles).

Sensor system 120 may include any suitable type of sensor. Various examples of different types of sensors will be described herein. However, it will be understood that the embodiments are not limited to the particular sensors described. Sensor system 120 may include one or more vehicle sensors 121. Vehicle sensor(s) 121 may detect, determine, sense, or acquire in a combination thereof information about vehicle 100 itself. In one or more arrangements, vehicle sensor(s) 121 may be configured to detect, sense, or acquire in a combination thereof position and orientation changes of vehicle 100, such as, for example, based on inertial acceleration. In one or more arrangements, vehicle sensor(s) 121 may include one or more accelerometers, one or more gyroscopes, an inertial measurement unit (IMU), a dead-reckoning system, a global navigation satellite system (GNSS), a global positioning system (GPS), a navigation system 147, other suitable sensors, or any combination thereof. Vehicle sensor(s) 121 may be configured to detect, sense, or acquire in a combination thereof one or more characteristics of vehicle 100. In one or more arrangements, vehicle sensor(s) 121 may include a speedometer to determine a current speed of vehicle 100.

Alternatively, or in addition, sensor system 120 may include one or more environment sensors 122 configured to acquire, sense, or acquire in a combination thereof driving environment data. “Driving environment data” includes data or information about the external environment in which an autonomous vehicle is located or one or more portions thereof. For example, environment sensor(s) 122 may be configured to detect, quantify, sense, or acquire in any combination thereof obstacles in at least a portion of the external environment of vehicle 100, information/data about such obstacles, or a combination thereof. Such obstacles may be comprised of stationary objects, dynamic objects, or a combination thereof. Environment sensor(s) 122 may be configured to detect, measure, quantify, sense, or acquire in any combination thereof other things in the external environment of vehicle 100, such as, for example, lane markers, signs, traffic lights, traffic signs, lane lines, crosswalks, curbs proximate to vehicle 100, off-road objects, etc.

Various examples of sensors of sensor system 120 will be described herein. The example sensors may be part of the one or more environment sensor(s) 122, the one or more vehicle sensors 121, or both. However, it will be understood that the embodiments are not limited to the particular sensors described.

As an example, in one or more arrangements, sensor system 120 may include one or more radar sensors 123, one or more LIDAR sensors 124, one or more sonar sensors 125, one or more cameras 126, or any combination thereof. In one or more arrangements, camera(s) 126 may be high dynamic range (HDR) cameras or infrared (IR) cameras.

Vehicle 100 may include an input system 130. An “input system” includes any device, component, system, element or arrangement or groups thereof that enable information/data to be entered into a machine. Input system 130 may receive an input from a vehicle passenger (e.g., a driver or a passenger). Vehicle 100 may include an output system 135. An “output system” includes any device, component, or arrangement or groups thereof that enable information/data to be presented to a vehicle passenger (e.g., a person, a vehicle passenger, etc.).

Vehicle 100 may include one or more vehicle systems 140. Various examples of vehicle system(s) 140 are shown in FIG. 1. However, vehicle 100 may include more, fewer, or different vehicle systems. It should be appreciated that although particular vehicle systems are separately defined, each or any of the systems or portions thereof may be otherwise combined or segregated via hardware, software, or a combination thereof within vehicle 100. Vehicle 100 may include a propulsion system 141, a braking system 142, a steering system 143, throttle system 144, a transmission system 145, a signaling system 146, a navigation system 147, other systems, or any combination thereof. Each of these systems may include one or more devices, components, or combinations thereof, now known or later developed.

Navigation system 147 may include one or more devices, applications, or combinations thereof, now known or later developed, configured to determine the geographic location of the vehicle 100, to determine a travel route for vehicle 100, or to determine both. Navigation system 147 may include one or more mapping applications to determine a travel route for vehicle 100. Navigation system 147 may include a global positioning system, a local positioning system, a geolocation system, or any combination thereof.

Processor(s) 110, AED management system 170, automated driving module(s) 160, or any combination thereof may be operatively connected to communicate with various aspects of vehicle system(s) 140 or individual components thereof. For example, returning to FIG. 1, processor(s) 110, automated driving module(s) 160, or a combination thereof may be in communication to send or receive information from various aspects of vehicle system(s) 140 to control the movement, speed, maneuvering, heading, direction, etc. of vehicle 100. Processor(s) 110, AED management system 170, automated driving module(s) 160, or any combination thereof may control some or all of these vehicle system(s) 140 and, thus, may be partially or fully autonomous.

Processor(s) 110, AED management system 170, automated driving module(s) 160, or any combination thereof may be operable to control at least one of the navigation or maneuvering of vehicle 100 by controlling one or more of vehicle systems 140 or components thereof. For instance, when operating in an autonomous mode, processor(s) 110, AED management system 170, automated driving module(s) 160, or any combination thereof may control the direction, speed, or both of vehicle 100. Processor(s) 110, AED management system 170, automated driving module(s) 160, or any combination thereof may cause vehicle 100 to accelerate (e.g., by increasing the supply of fuel provided to the engine), decelerate (e.g., by decreasing the supply of fuel to the engine, by applying brakes), change direction (e.g., by turning the front two wheels), or perform any combination thereof. As used herein, “cause” or “causing” means to make, force, compel, direct, command, instruct, enable, or in any combination thereof an event or action to occur or at least be in a state where such event or action may occur, either in a direct or indirect manner.

Vehicle 100 may include one or more actuators 150. Actuator(s) 150 may be any element or combination of elements operable to modify, adjust, alter, or in any combination thereof one or more of vehicle systems 140 or components thereof to responsive to receiving signals or other inputs from processor(s) 110, automated driving module(s) 160, or a combination thereof. Any suitable actuator may be used. For instance, actuator(s) 150 may include motors, pneumatic actuators, hydraulic pistons, relays, solenoids, and piezoelectric actuators, just to name a few possibilities.

Vehicle 100 may include one or more modules, at least some of which are described herein. The modules may be implemented as computer-readable program code that, when executed by processor(s) 110, implement one or more of the various processes described herein. One or more of the modules may be a component of processor(s) 110, or one or more of the modules may be executed on or distributed among other processing systems to which processor(s) 110 is operatively connected. The modules may include instructions (e.g., program logic) executable by processor(s) 110. Alternatively, or in addition, data store(s) 115 may contain such instructions.

In one or more arrangements, one or more of the modules described herein may include artificial or computational intelligence elements, e.g., neural network, fuzzy logic, or other machine learning algorithms. Further, in one or more arrangements, one or more of the modules may be distributed among a plurality of the modules described herein. In one or more arrangements, two or more of the modules described herein may be combined into a single module.

Vehicle 100 may include one or more autonomous driving module(s) 160. Automated driving module(s) 160 may be configured to receive data from sensor system 120 or any other type of system capable of capturing information relating to vehicle 100, the external environment of the vehicle 100, or a combination thereof. In one or more arrangements, automated driving module(s) 160 may use such data to generate one or more driving scene models. Automated driving module(s) 160 may determine position and velocity of vehicle 100. Automated driving module(s) 160 may determine the location of obstacles, obstacles, or other environmental features including traffic signs, trees, shrubs, neighboring vehicles, pedestrians, etc.

Automated driving module(s) 160 may be configured to receive, determine, or in a combination thereof location information for obstacles within the external environment of vehicle 100, which may be used by processor(s) 110, one or more of the modules described herein, or any combination thereof to estimate: a position or orientation of vehicle 100; a vehicle position or orientation in global coordinates based on signals from a plurality of satellites or other geolocation systems; or any other data/signals that could be used to determine a position or orientation of vehicle 100 with respect to its environment for use in either creating a map or determining the position of vehicle 100 in respect to map data.

Automated driving module(s) 160 either independently or in combination with AED management system 170may be configured to determine travel path(s), current autonomous driving maneuvers for vehicle 100, future autonomous driving maneuvers, modifications to current autonomous driving maneuvers, etc. Such determinations by automated driving module(s) 160 may be based on data acquired by sensor system 120, driving scene models, data from any other suitable source such as determinations from sensor data 260, or any combination thereof. In general, automated driving module(s) 160 may function to implement different levels of automation, including advanced driving assistance (ADAS) functions, semi-autonomous functions, and fully autonomous functions. “Driving maneuver” means one or more actions that affect the movement of a vehicle. Examples of driving maneuvers include accelerating, decelerating, braking, turning, moving in a lateral direction of vehicle 100, changing travel lanes, merging into a travel lane, and reversing, just to name a few possibilities. Automated driving module(s) 160 may be configured to implement driving maneuvers. Automated driving module(s) 160 may cause, directly or indirectly, such autonomous driving maneuvers to be implemented. As used herein, “cause” or “causing” means to make, command, instruct, enable, or in any combination thereof an event or action to occur or at least be in a state where such event or action may occur, either in a direct or indirect manner. Automated driving module(s) 160 may be configured to execute various vehicle functions, whether individually or in combination, to transmit data to, receive data from, interact with, or to control vehicle 100 or one or more systems thereof (e.g., one or more of vehicle systems 140).

Detailed embodiments are disclosed herein. However, it is to be understood that the disclosed embodiments are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various embodiments are shown in FIGS. 1-5, but the embodiments are not limited to the illustrated structure or application.

The flowcharts and block diagrams in the figures illustrate the architecture, functionality, and operation of possible implementations of systems, methods, and computer program products according to various embodiments. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components, or processes described above may be realized in hardware or a combination of hardware and software and may be realized in a centralized fashion in one processing system or in a distributed fashion where different elements are spread across several interconnected processing systems. Any kind of processing system or another apparatus adapted for carrying out the methods described herein is suited. A typical combination of hardware and software may be a processing system with computer-usable program code that, when being loaded and executed, controls the processing system such that it carries out the methods described herein. The systems, components, or processes also may be embedded in a computer-readable storage, such as a computer program product or other data programs storage device, readable by a machine, tangibly embodying a program of instructions executable by the machine to perform methods and processes described herein. These elements also may be embedded in an application product which comprises all the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a read-only memory (ROM), an erasable programmable read-only memory (EPROM or Flash memory), a portable compact disc read-only memory (CD-ROM), a digital versatile disc (DVD), an optical storage device, a magnetic storage device, or any suitable combination of the foregoing. In the context of this document, a computer-readable storage medium may be any tangible medium that may contain or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an application-specific integrated circuit (ASIC), a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, RF, etc., or any suitable combination of the foregoing. Computer program code for carrying out operations for aspects of the present arrangements may be written in any combination of one or more programming languages, including an object-oriented programming language such as Java™, Smalltalk, C++, or the like and conventional procedural programming languages, such as the “C” programming language or similar programming languages. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The terms “a” and “an,” as used herein, are defined as one or more than one. The term “plurality,” as used herein, is defined as two or more than two. The term “another,” as used herein, is defined as at least a second or more. The terms “including” and “having,” as used herein, are defined as comprising (i.e., open language). The phrase “at least one of . . . and . . . ” as used herein refers to and encompasses any and all possible combinations of one or more of the associated listed items. As an example, the phrase “at least one of A, B, and C” includes A only, B only, C only, or any combination thereof (e.g., AB, AC, BC, or ABC).

Aspects herein may be embodied in other forms without departing from the spirit or essential attributes thereof. Accordingly, reference should be made to the following claims, rather than to the foregoing specification, as indicating the scope hereof.

What is claimed is:

1. A system, comprising:
a processor; and
a memory communicably coupled to the processor and storing machine-readable instructions that, when executed by the processor, cause the processor to:
determine if a low-voltage power component lacks power to generate an electric shock signal; and
generate the electric shock signal via a high-voltage power component if the low-voltage power component lacks power to generate the electric shock signal.

2. The system of claim 1, wherein the machine-readable instructions further includes to provide backup operating power via the high-voltage power component.

3. The system of claim 1, wherein the machine-readable instructions further includes to generate the electric shock signal via the low-voltage power component if the low-voltage power component has power to generate the electric shock signal.

4. The system of claim 1, wherein the machine-readable instructions to generate the electric shock signal does not occur if an access request is not validated.

5. The system of claim 4, wherein the machine-readable instructions further includes to:
request that a validation action be performed; and
validate the access request if a record indicates that the validation action has been performed.

6. The system of claim 1, wherein the machine-readable instructions to generate the electric shock signal is implemented with a buck-boost converter.

7. The system of claim 1, wherein the machine-readable instructions to generate the electric shock signal is implemented with a wideband gap material.

8. A non-transitory computer-readable medium including instructions that when executed by one or more processors cause the one or more processors to:
determine if a low-voltage power component lacks power to generate an electric shock signal; and
generate the electric shock signal via a high-voltage power component if the low-voltage power component lacks power to generate the electric shock signal.

9. The non-transitory computer-readable medium of claim 8, wherein the instructions further include to provide backup operating power via the high-voltage power component.

10. The non-transitory computer-readable medium of claim 8, wherein the instructions further include to generate the electric shock signal via the low-voltage power component if the low-voltage power component has power to generate the electric shock signal.

11. The non-transitory computer-readable medium of claim 8, wherein the instruction to generate the electric shock signal does not occur if an access request is not validated.

12. The non-transitory computer-readable medium of claim 11, wherein the instruction to:
request that a validation action be performed; and
validate the access request if a record indicates that the validation action has been performed.

13. The non-transitory computer-readable medium of claim 8, wherein the instruction to generate the electric shock signal is implemented with a buck-boost converter.

14. A method, comprising:
determining if a low-voltage power component lacks power to generate an electric shock signal; and
generating the electric shock signal via a high-voltage power component if the low-voltage power component lacks power to generate the electric shock signal.

15. The method of claim 14, further comprising providing backup operating power via the high-voltage power component.

16. The method of claim 14, further comprising generating the electric shock signal via the low-voltage power component if the low-voltage power component has power to generate the electric shock signal.

17. The method of claim 14, wherein generating the electric shock signal does not occur if an access request is not validated.

18. The method of claim 17, further comprising:
requesting that a validation action be performed; and
validating the access request if a record indicates that the validation action has been performed.

19. The method of claim 14, wherein generating the electric shock signal is implemented with a buck-boost converter.

20. The method of claim 14, wherein generating the electric shock signal is implemented with a wideband gap material.
